(19) United States
(12) Patent Application Publication
(10) Pub. No.: US 2023/0301659 A1
(43) Pub. Date: Sep. 28, 2023

(54) MEDICAL SUPPORT SYSTEM, METHOD FOR CONTROLLING MEDICAL SUPPORT SYSTEM, AND MEDICAL SUPPORT METHOD

(71) Applicant: TERUMO KABUSHIKI KAISHA, Tokyo (JP)

(72) Inventors: Kaori ENOMOTO, Fujisawa-shi (JP); Itaru OKUBO, Ninomiya-cho (JP); Yoshiharu EZASHI, Nakai-machi (JP)

(73) Assignee: TERUMO KABUSHIKI KAISHA, Tokyo (JP)

(21) Appl. No.: 18/169,500

(22) Filed: Feb. 15, 2023

(30) Foreign Application Priority Data
Mar. 28, 2022 (JP) 2022-051512

(51) Int. Cl. A61B 17/12; A61B 5/02

(52) U.S. Cl. CPC A61B 17/12113; A61B 5/02007; A61B 17/1214; A61B 2017/00022

(57) ABSTRACT

A medical support system that supports intravascular treatment includes: a detection device inserted into a blood vessel and configured to be capable of detecting a state of a treatment target which is the blood vessel or an object attached to the blood vessel; and a control unit configured to select a treatment device or a treatment method, which is suitable for the state of the treatment target detected by the detection device.

### CROSS-REFERENCES TO RELATED APPLICATIONS

This application claims priority to Japanese Application No. 2022-051512 filed on Mar. 28, 2022, the entire content of which is incorporated herein by reference.

### TECHNOLOGICAL FIELD

The present disclosure generally relates to a medical support system that supports selection of a device and a treatment method, which are appropriate for intravascular treatment, a method of controlling a medical support system, and a medical support method.

### BACKGROUND DISCUSSION

As a treatment device for an aneurysm such as a cerebral aneurysm, in recent years, an embolic coil packed in the aneurysm (refer to, for example, WO 2021/065274 A) or a tubular flow diverter stent having a fine mesh covering an inner surface of an artery in which the aneurysm is formed has been used. The embolic coil and the flow diverter stent have been increasingly used in recent years, and it has been reported that the rate of post-operative complications according to surgical operations (clipping operations) are almost the same.

The complete occlusion rate of the aneurysm of a patient subjected to intravascular treatment is about 60%, and a certain number of patients require treatment again after the intravascular treatment. In order to increase the complete occlusion rate of the aneurysm, it is preferable to place a coil having a shape more suitable for each aneurysm. However, it is difficult to select an appropriate coil since information regarding the hardness of an aneurysm wall cannot be obtained by obtaining the aneurysm diameter and aneurysm shape by using the current contrast agent.

Similarly, even in the aspiration removal of a thrombus in intravascular stenosis such as a cerebral infarction and lower limb stenosis, it is necessary to capture the thrombus with an appropriate force or aspirate the thrombus. However, there is a demand for a method capable of selecting a stent retriever having an appropriate dimension or selecting an appropriate aspiration force in advance from the hardness of the thrombus or blood vessel wall.

### SUMMARY

A medical support system, a method of controlling the medical support system, and a medical support method are disclosed, which are capable of selecting an appropriate treatment device or treatment method according to a blood vessel in a different state or an object attached to the blood vessel.

A medical support system is disclosed that supports intravascular treatment, the medical support system including: a detection device inserted into a blood vessel and configured to be capable of detecting a state of a treatment target which is the blood vessel or an object attached to the blood vessel; and a control unit configured to select a treatment device or a treatment method, which is suitable for the state of the treatment target detected by the detection device.

In the medical support system configured as described above, the state of the blood vessel or the state of the object attached to the blood vessel is quantified, and the control unit selects an appropriate treatment device or treatment method according to the numerical value. Therefore, the medical support system can select the appropriate treatment device or treatment method according to the blood vessel in a different state or the object attached to the blood vessel.

The detection device may include an inflation portion capable of inflating and deflating in the blood vessel, a contact detection unit configured to detect contact of the inflation portion with the treatment target, and a strain detection unit configured to detect a strain amount of the treatment target. The control unit may receive a contact detection result from the contact detection unit and a strain amount detection result from the strain detection unit, calculate flexibility or elasticity which is a state of the treatment target from the contact detection result and the strain amount detection result, and select the treatment device or treatment method on the basis of the calculated flexibility or elasticity. Therefore, the medical support system can accurately detect the flexibility or elasticity of the treatment target, and thus it is possible to select an appropriate treatment device or treatment method.

The control unit is capable of receiving an image from an imaging apparatus capable of capturing the image with which a size of the treatment target is capable of being specified. The control unit may determine whether or not a strain ratio, which is a ratio of an absolute value of a deviation amount of an actual strain amount detected by the strain detection unit, with respect to an assumed strain amount which is a strain amount assumed to be generated in the treatment target in a case where a predetermined inflation force is applied to the inflation portion, is within a preset allowable value on an assumption that the treatment target includes a reference compliance value set in advance, and determine the treatment device or treatment method calculated from the size of the treatment target specified from the image acquired from the imaging apparatus as a treatment device or treatment method to be selected, in a case where the strain ratio is within the allowable value; and the control unit may calculate an actual compliance value of the treatment target from the strain amount measured by the strain detection unit to determine whether the compliance value is greater than or less than the reference compliance value in a case where a predetermined inflation force is applied to the inflation portion after the contact detection unit detects the contact of the inflation portion with the treatment target in a case where the strain ratio exceeds the allowable value, determine a treatment device or treatment method capable of obtaining a coil packing ratio higher than the coil packing ratio of the treatment device or treatment method calculated from the size of the treatment target specified from the image acquired from the imaging apparatus, as a treatment device or treatment method to be selected, in a case where the actual compliance value is greater than the reference compliance value, and determine a treatment device or treatment method capable of obtaining a coil packing ratio lower than the coil packing ratio of the treatment device or treatment method calculated from the size of the treatment target specified from the image acquired from the imaging apparatus, as a treatment device or treatment method to be selected, in a case where the actual compliance value is less than the reference compliance value. Therefore, the medical support system can select an appropriate treatment device or treatment method by using both the size of the treatment target that can be specified from the image obtained from the imaging apparatus and the measured compliance value of the treatment target. The present medical support system can determine a treatment method such as performing determination that the high coil packing ratio can be obtained even when some force is applied in a case where the actual compliance value is great and the treatment target is soft, or performing determination that the low coil packing ratio can be obtained such that the coil is not excessively packed in a case where the actual compliance value is relatively small and the treatment target is relatively hard.

A method for controlling a medical support system that supports intravascular treatment, the method including: receiving a detection result from a detection device inserted into a blood vessel and configured to be capable of detecting a state of a treatment target which is the blood vessel or an object attached to the blood vessel; selecting a treatment device or treatment method suitable for the state of the treatment target detected by the detection device; and transmitting information regarding the selected treatment device or treatment method to a display unit capable of displaying the information to cause the display unit to display the information.

In the method for controlling a medical support system configured as described above, the state of the blood vessel or the state of the object attached to the blood vessel is quantified, and an appropriate treatment device or treatment method according to the numerical value can be selected to be displayed on the display unit. Therefore, in the method for controlling a medical support system, the appropriate treatment device or treatment method according to the blood vessel in a different state or the object attached to the blood vessel can be selected to be displayed.

The receiving of the detection result may include receiving a contact detection result from a contact detection unit configured to detect contact of an inflation portion capable of inflating and deflating in the blood vessel with the treatment target, and receiving a strain amount detection result from a strain detection unit configured to detect a strain amount of the treatment target, and the selecting of the treatment device or treatment method may include calculating flexibility or elasticity which is a state of the treatment target from the contact detection result and the strain amount detection result, and selecting the treatment device or treatment method on the basis of the calculated flexibility or elasticity. Therefore, the method for controlling a medical support system can accurately detect the flexibility or elasticity of the treatment target, and thus it is possible to control the medical support system so as to select an appropriate treatment device or treatment method.

The selecting of the treatment device or treatment method may include: determining whether or not a strain ratio, which is a ratio of an absolute value of a deviation amount of an actual strain amount detected by the strain detection unit, with respect to an assumed strain amount which is a strain amount assumed to be generated in the treatment target in a case where a predetermined inflation force is applied to the inflation portion, is within a preset allowable value on an assumption that the treatment target includes a reference compliance value set in advance; determining the treatment device or treatment method calculated from a size of the treatment target specified from an image acquired from an imaging apparatus configured to be capable of capturing an image of the treatment target as a treatment device or treatment method to be selected, in a case where the strain ratio is within the allowable value; calculating an actual compliance value of the treatment target from the strain amount measured by the strain detection unit to determine whether the compliance value is greater than or less than the reference compliance value when a predetermined inflation force is applied to the inflation portion in a case where the strain ratio exceeds the allowable value; determining a treatment device or treatment method capable of obtaining a coil packing ratio higher than the coil packing ratio of the treatment device or treatment method calculated from the size of the treatment target specified from the image acquired from the imaging apparatus, as a treatment device or treatment method to be selected, in a case where the actual compliance value is greater than the reference compliance value; and determining a treatment device or treatment method capable of obtaining a coil packing ratio lower than the coil packing ratio of the treatment device or treatment method calculated from the size of the treatment target specified from the image acquired from the imaging apparatus, as a treatment device or treatment method to be selected, in a case where the actual compliance value is less than the reference compliance value. Therefore, the method for controlling a medical support system can control the medical support system so as to select an appropriate treatment device or treatment method by using both the size of the treatment target that can be specified from the image obtained from the imaging apparatus and the measured compliance value of the treatment target. In the present control method, a treatment method can be determined, such as performing determination that the high coil packing ratio can be obtained even when some force is applied in a case where the actual compliance value is great and the treatment target is soft, or performing determination that the low coil packing ratio can be obtained such that the coil is not excessively packed in a case where the actual compliance value is relatively small and the treatment target is relatively hard.

A medical support method is disclosed that supports intravascular treatment, the method including: inserting, into a blood vessel, a detection device configured to be capable of detecting a state of a treatment target which is the blood vessel or an object attached to the blood vessel to detect the state of the treatment target; selecting a treatment device or treatment method suitable for the state of the treatment target detected by the detection device; and displaying information regarding the selected treatment device or treatment method on a display unit configured to be capable of displaying the information.

In the medical support method configured as described above, the state of the blood vessel or the state of the object attached to the blood vessel is quantified, and an appropriate treatment device or treatment method according to the numerical value can be selected to be displayed on the display unit. Therefore, in the medical support method, the appropriate treatment device or treatment method according to the blood vessel in a different state or the object attached to the blood vessel can be selected to be displayed.

The detecting of the state of the treatment target may include detecting contact of an inflation portion capable of inflating and deflating in the blood vessel with the treatment target and detecting a strain amount of the treatment target, and the selecting of the treatment device or treatment method may include calculating flexibility or elasticity which is a state of the treatment target from a contact detection result and a strain amount detection result, and selecting the treatment device or treatment method on the basis of the calculated flexibility or elasticity of the treatment target. Therefore, in the medical support method, the flexibility or elasticity of the treatment target can be accurately detected, and thus it is possible to select an appropriate treatment device or treatment method.

The selecting of the treatment device or treatment method may include: determining whether or not a strain ratio, which is a ratio of an absolute value of a deviation amount of an actual strain amount detected by a strain detection unit, with respect to an assumed strain amount which is a strain amount assumed to be generated in the treatment target in a case where a predetermined inflation force is applied to the inflation portion, is within a preset allowable value on an assumption that the treatment target includes a reference compliance value set in advance; determining the treatment device or treatment method calculated from a size of the treatment target specified from an image acquired from an imaging apparatus configured to be capable of capturing an image of the treatment target as a treatment device or treatment method to be selected, in a case where the strain ratio is within the allowable value; calculating an actual compliance value of the treatment target from the strain amount measured by the strain detection unit to determine whether the compliance value is greater than or less than the reference compliance value when a predetermined inflation force is applied to the inflation portion in a case where the strain ratio exceeds the allowable value; determining a treatment device or treatment method capable of obtaining a coil packing ratio higher than the coil packing ratio of the treatment device or treatment method calculated from the size of the treatment target specified from the image acquired from the imaging apparatus, as a treatment device or treatment method to be selected, in a case where the actual compliance value is greater than the reference compliance value; and determining a treatment device or treatment method capable of obtaining a coil packing ratio lower than the coil packing ratio of the treatment device or treatment method calculated from the size of the treatment target specified from the image acquired from the imaging apparatus, as a treatment device or treatment method to be selected, in a case where the actual compliance value is less than the reference compliance value. Therefore, in the medical support method, an appropriate treatment device or treatment method can be selected by using both the size of the treatment target that can be specified from the image obtained from the imaging apparatus and the measured compliance value of the treatment target. In the present medical support method, a treatment method can be determined, such as performing determination that the high coil packing ratio can be obtained even when some force is applied in a case where the actual compliance value is great and the treatment target is soft, or performing determination that the low coil packing ratio can be obtained such that the coil is not excessively packed in a case where the actual compliance value is relatively small and the treatment target is relatively hard.

### BRIEF DESCRIPTION OF THE DRAWINGS

FIG. 1 is a schematic front view illustrating a medical support system according to an embodiment.

FIG. 2 is a configuration diagram illustrating a medical support system according to the embodiment.

FIG. 3 is a cross-sectional view illustrating a distal end portion of a detection device.

FIG. 4 is a flowchart illustrating a flow of control in a control unit.

FIGS. 5A and 5B are schematic explanatory views illustrating a state in which a detection device is inserted into a blood vessel, FIG. 5A illustrates a state in which a balloon is inserted into an aneurysm, and FIG. 5B illustrates a state in which the balloon is inflated in the aneurysm.

### DETAILED DESCRIPTION

Set forth below with reference to the accompanying drawings is a detailed description of embodiments of a medical support system that supports selection of a device and a treatment method, which are appropriate for intravascular treatment, a method of controlling a medical support system, and a medical support method. Note that since embodiments described below are preferred specific examples of the present disclosure, although various technically preferable limitations are given, the scope of the present disclosure is not limited to the embodiments unless otherwise specified in the following descriptions. The dimensions of the drawings may be exaggerated and different from actual dimensions for convenience of description. In the present specification and the drawings, components having substantially the same functional configuration are denoted by the same reference numerals, and the overlapping description will be omitted. In the present specification, a side to be inserted into a lumen is referred to as a “distal end side”, and a side of a hand operation portion is referred to as a “base end side”.

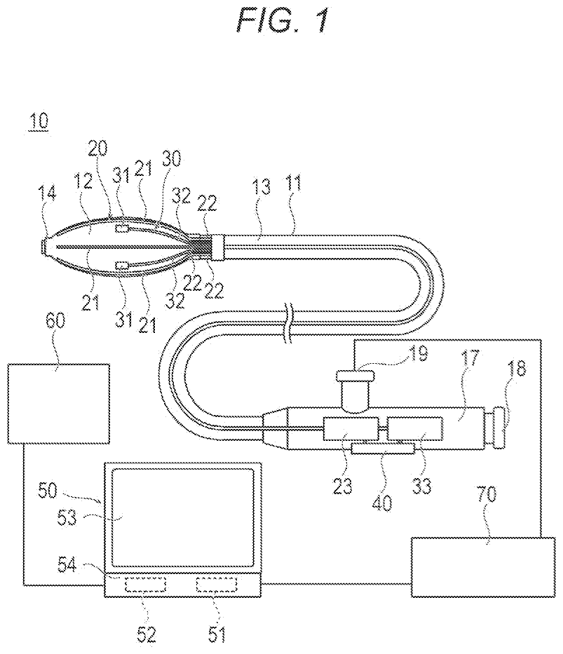

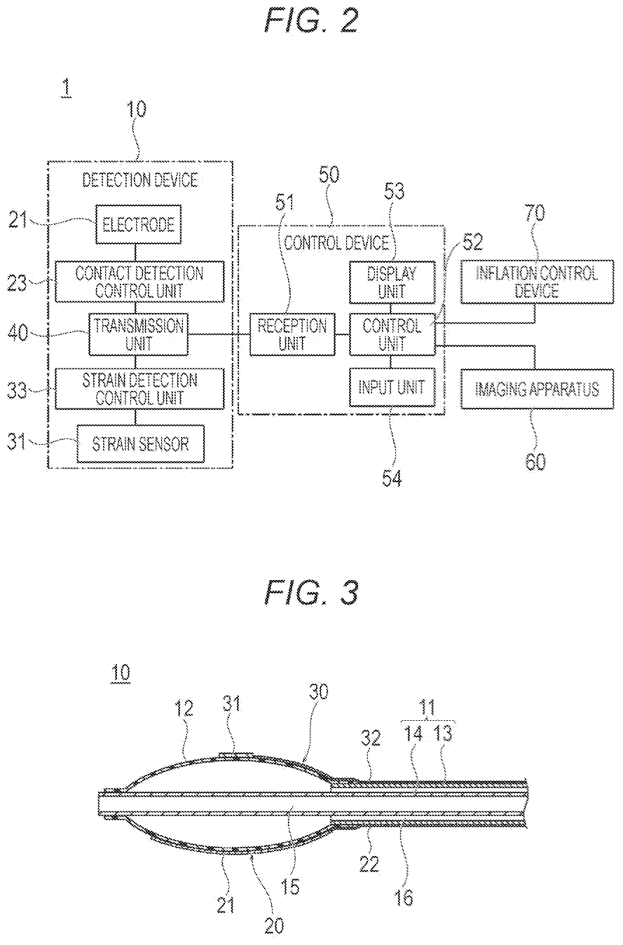

A medical support system 1 according to the present embodiment is a system that detects flexibility (hardness) or elasticity (suppleness), which is a state of a tissue of an aneurysm, automatically selects a treatment device and treatment method suitable for the state, and notifies an operator of the selected treatment device and treatment method. As illustrated in FIGS. 1 and 2, the medical support system 1 can include a detection device 10 that is inserted into a blood vessel and detects flexibility of the tissue of an aneurysm, an inflation control device 70 that supplies a fluid for inflating a balloon 12 (inflation portion) of the detection device 10 to the detection device 10, a control device 50 that receives a detection result from the detection device 10, automatically selects a treatment device (for example, an embolic coil) suitable for the detected state of the tissue, and notifies the operator of the selected treatment device, and an imaging apparatus 60.

The detection device 10 is an elongated balloon catheter system that is percutaneously inserted into a blood vessel and pushed into the aneurysm. As illustrated in FIGS. 1 to 3, the detection device 10 can include an elongated shaft portion 11, a balloon 12 that is an inflation portion provided at a distal end portion of the shaft portion 11, a hub 17 fixed to a base end portion of the shaft portion 11, a contact detection unit 20 that detects contact of the balloon 12 with an inner wall surface of the aneurysm, a strain detection unit 30 that detects a strain amount of the tissue of the aneurysm, and a transmission unit 40 that transmits information to the control device 50.

The shaft portion 11 can include an outer tube 13 and an inner tube 14 disposed coaxially inside the outer tube 13. A guide wire lumen 15 extending in a length direction is formed inside the inner tube 14. A guide wire can be inserted into the guide wire lumen 15. An inflation lumen 16 is formed inside the outer tube 13 and outside the inner tube 14. An inflation fluid for inflating the balloon 12 can flow through the inflation lumen 16. The inflation fluid may be gas or liquid, and for example, the gas such as helium gas, CO₂ gas, O₂ gas, or laughing gas, or the liquid such as normal saline solution (saline solution), a contrast agent, or a mixture of normal saline solution and a contrast agent can be used.

The balloon 12 is a tubular body that is flexibly deformable. The balloon 12 can be inflated in the aneurysm to come into contact with the inner wall surface of the aneurysm, and can further expand the aneurysm. A distal end portion of the balloon 12 is fixed to a distal end portion of the inner tube 14, and a base end portion of the balloon 12 is fixed to a distal end portion of the outer tube 13. The balloon 12 can have a thin film shape, is inflatable when the fluid is supplied to the inside through the inflation lumen 16, and is deflatable when the fluid in the balloon 12 is discharged through the inflation lumen 16. The balloon 12 can be formed of or fabricated from, for example, a resin such as silicone or an elastomer, which is flexibly stretchable.

A base end portion of the shaft portion 11 is fixed to the hub 17. The hub 17 can include a first port 18 having an opening communicating with the guide wire lumen 15 and a second port 19 having an opening communicating with the inflation lumen 16.

The contact detection unit 20 can include a plurality of electrodes 21 disposed on the outer surface of the balloon 12, a plurality of electrode conductive wires 22 connected to the electrodes 21, and a contact detection control unit 23 connected to the electrode conductive wires 22 to supply power to a plurality of the electrodes 21 and detecting impedance between the electrodes 21. A plurality of the electrodes 21 are preferably disposed at a plurality of positions on the outer surface of the balloon 12 so as to detect that substantially the entire outer surface of the balloon 12 is in contact with the inner wall surface of the aneurysm. The electrodes 21 formed by pairing positive and negative electrodes for causing a current to flow may be disposed side by side in a circumferential direction of the balloon 12 or may be disposed side by side in an axial direction of the balloon 12. The contact detection control unit 23 can be fixed to, for example, the hub 17. A plurality of the electrode conductive wires 22 extend from the electrodes 21 to the contact detection control unit 23. It is preferable that a plurality of the electrode conductive wires 22 are disposed along the shaft portion 11 as one bundle (i.e., the plurality of electrodes conductive wires 22 are collected together).

The strain detection unit 30 can include a plurality of strain sensors 31 disposed on the outer surface of the balloon 12, a plurality of strain sensor conductive wires 32 respectively connected to the strain sensors 31, and a strain detection control unit 33 connected to the strain sensor conductive wires 32 to supply power to a plurality of strain sensors 31 and detecting outputs from the strain sensors 31. Each of the strain sensors 31 can detect the strain amount of the aneurysm when the balloon 12 is deformed together with the object (herein, the aneurysm) with which the balloon 12 comes into contact. A plurality of the strain sensors 31 are disposed at a plurality of positions on the outer surface of the balloon 12 such that the strain sensors 31 can detect the strain amount of substantially the entire inner wall surface of the aneurysm in contact with substantially the entire outer surface of the balloon 12. A plurality of the strain sensors 31 may be disposed side by side in a circumferential direction of the balloon 12 or may be disposed side by side in an axial direction of the balloon 12. The strain detection control unit 33 can be fixed to, for example, the hub 17. A plurality of the strain sensor conductive wires 32 extend from the strain sensor 31 to the strain detection control unit 33. It is preferable that a plurality of the strain sensor conductive wires 32 are disposed along the shaft portion 11 as one bundle.

The transmission unit 40 receives an input of a contact detection result, which is a detection result for the impedance between the electrodes 21, from the contact detection control unit 23, and wirelessly transmits the contact detection result to the control device 50. The transmission unit 40 receives an input of a distortion amount detection result, which is a detection result of the strain sensor 31, from the strain detection control unit 33, and wirelessly transmits the strain amount detection result to the control device 50. The transmission unit 40 may transmit the contact detection result and the strain amount detection result to the control device 50 not wirelessly but by wire.

The inflation control device 70 is connected to the second port 19 of the hub 17 of the detection device 10, and thus can supply a fluid for inflating the balloon 12. The inflation control device 70 can be, for example, a syringe pump, a tube pump, an indeflator, or the like that can supply a fluid by controlling a supply pressure or a supply volume. The operation of the inflation control device 70 is controlled by the control device 50.

The imaging apparatus 60 is a device capable of capturing a three-dimensional image (a plurality of cross-sectional images) of the inside of the patient's body being subjected to a procedure, and can be, for example, a contrast-enhanced computed tomography (CT) apparatus, a magnetic resonance imaging (MRI) apparatus, a magnetic resonance angiography (MRA) apparatus, or the like. The imaging apparatus 60 can transmit electronic data of the captured image to the control device 50.

The control device 50 can include a reception unit 51 that receives a signal from the transmission unit 40, a control unit 52 that performs arithmetic processing, a display unit 53 such as a monitor that displays information as an image, and an input unit 54 such as a touch panel, a keyboard, and/or a mouse. At least a part of the control device 50 can be formed by, for example, a computer.

The reception unit 51 can transmit a signal for controlling the contact detection control unit 23 and the strain detection control unit 33 to the transmission unit 40. The reception unit 51 can receive a wireless signal indicating the contact detection result and strain amount detection result from the transmission unit 40 and transmit the wireless signal to an arithmetic unit (or arithmetic logic unit).

The control unit 52 can include a storage circuit and an arithmetic circuit. The storage circuit can store programs, various kinds of parameters, and various kinds of data. The arithmetic unit can read the programs, the various kinds of parameters, and the various kinds of data from the storage circuit, and perform arithmetic processing. The arithmetic circuit can be, for example, a central processing unit (CPU). The control unit 52 receives a signal indicating the contact detection result and strain amount detection result from the reception unit 51. The control unit 52 can control the operation of the imaging apparatus 60 and receive data of an image of a patient from the imaging apparatus 60. The control unit 52 can control the operation of the inflation control device 70 and receive information (for example, information regarding a supply pressure and a supply volume) from the inflation control device 70.

The control unit 52 can perform arithmetic processing of selecting specifications (for example, a manufacturer, a model number, a loop diameter, a coil length, and the like) of the embolic coil appropriate for embolization of the aneurysm, a treatment method, and the like on the basis of the received contact detection result and strain amount detection result. The arithmetic processing in the control unit 52 will be described in detail later.

The display unit 53 can receive the specifications of the embolic coil selected by the control unit 52, the treatment method, and the like from the control unit 52, display the specifications and the treatment method, and notify the operator of the specifications and the treatment method. The input unit 54 can be used for the operator to perform operations such as input and various settings.

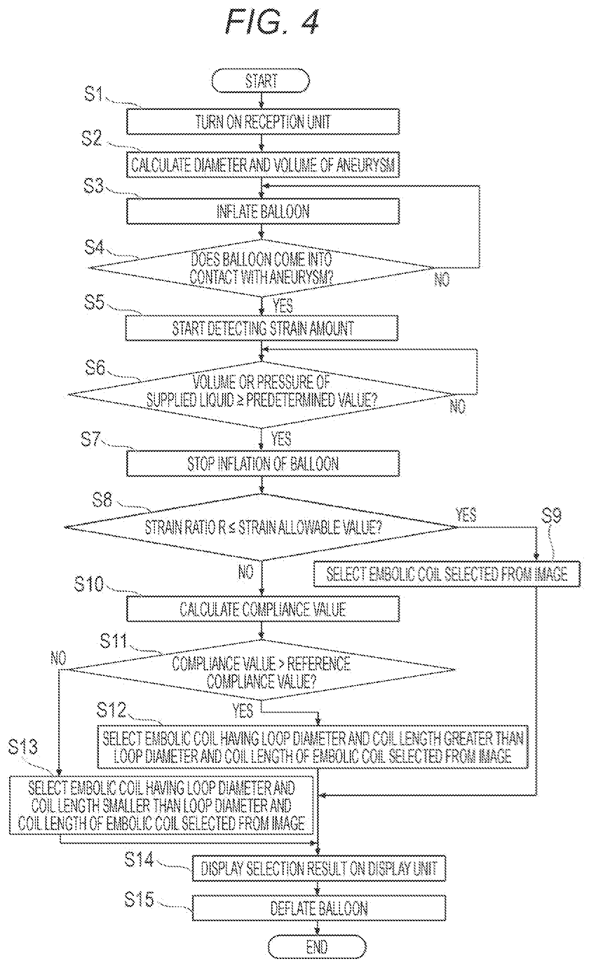

Next, the operation control of the control unit 52 will be described with reference to the flowchart illustrated in FIG. 4.

The operator operates the input unit 54 of the control device 50 to cause the control unit 52 to start control operation. The control unit 52 activates the reception unit 51 to start communication with the treatment device (S1).

Next, the operator can inject a contrast agent into a blood vessel. The control unit 52 acquires an image having a range including the aneurysm from the imaging apparatus 60, and calculates the diameter and internal volume of the aneurysm from the image (S2).

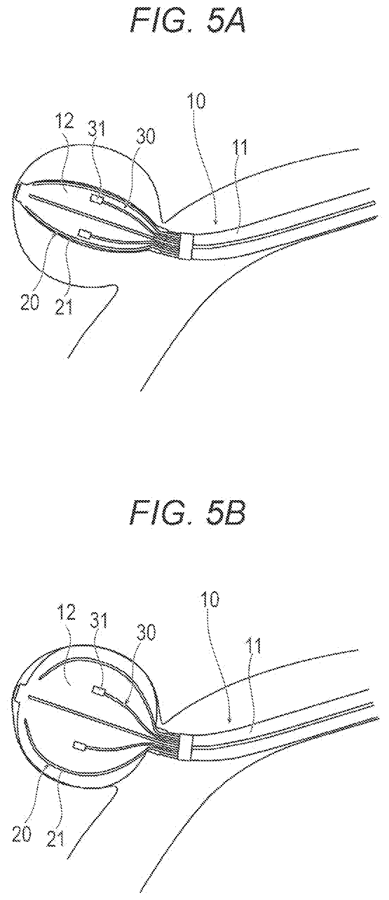

Next, the operator inserts the detection device 10 percutaneously into the blood vessel, pushes the detection device 10 while appropriately injecting the contrast agent, and inserts the balloon 12 into the aneurysm as illustrated in FIG. 5A. Next, the control unit 52 controls the inflation control device 70 to supply the fluid to the detection device 10, and inflates the balloon 12 as illustrated in FIG. 5B (S3). The fluid flows from the second port 19 of the hub 17 of the detection device 10 into the inside of the balloon 12 through the inflation lumen 16 and thus the balloon 12 is inflated. The control unit 52 starts supply of the fluid and detection of impedance between the electrodes 21 of the contact detection unit 20 disposed in the balloon 12.

The control unit 52 receives the contact detection result obtained from the electrodes 21, which is transmitted from the transmission unit 40 of the treatment device, via the reception unit 51. The control unit 52 monitors the contact detection result, determines that the outer surface of the balloon 12 is in close contact with the inner wall surface of the aneurysm when detecting a change in impedance at all the detection points on the outer surface of the balloon 12 (decrease in impedance due to contact of the electrodes 21 with the inner wall surface of the aneurysm) (S4), and stops the supply of the fluid by the inflation control device 70.

Next, the control unit 52 causes a plurality of the strain sensors 31 disposed on the outer surface of the balloon 12 to start detection, and receives a strain amount c, which is a strain amount detection result, via the reception unit 51 (S5). Next, in order to apply a predetermined inflation force of the balloon 12 to the aneurysm of the treatment target, the control unit 52 controls the inflation control device 70 to slowly supply the fluid until the volume of the supplied fluid reaches a predetermined value, and thus slowly inflates the balloon 12 (S6). Alternatively, in order to apply a predetermined inflation force of the balloon 12 to the aneurysm of the treatment target, the control unit 52 may control the inflation control device 70 to slowly supply the fluid until the pressure of the fluid increases to a predetermined value, and thus may slowly inflate the balloon 12. The predetermined value of the pressure or volume of the fluid is determined in consideration of the internal volume of the aneurysm calculated from the image and the volume of the range surrounded by the balloon 12 when it is determined that the outer surface of the balloon 12 is in close contact with the inner wall of the aneurysm. The predetermined value of the pressure or volume of the fluid may be preset. The predetermined value of the pressure or volume of the fluid is preferably set such that the balloon 12 does not inflate to exceed the internal volume of the aneurysm in consideration of safety.

As described above, in order to apply a predetermined inflation force of the balloon 12 to the aneurysm, the control unit 52 stops the inflation of the balloon 12 after the fluid is supplied until the volume of the supplied fluid reaches a predetermined value or after the fluid is supplied until the pressure of the fluid increases by the predetermined value (S7).

Next, in S6, the control unit 52 calculates the strain amount ε until the inflation of the balloon 12 is stopped after the inflation of the balloon 12 is restarted. That is, the control unit 52 calculates the strain amount ε generated when a predetermined inflation force of the balloon 12 is applied to the aneurysm. The control unit 52 calculates, as an assumed strain amount ε0, a strain amount assumed in a case where it is assumed that the aneurysm has a reference compliance value (compliance value of a general aneurysm). The compliance value is an index indicating flexibility or elasticity (elongation or suppleness) of the tissue. Subsequently, in a case where the assumed strain amount ε0 is 100%, the control unit 52 determines whether or not a strain ratio R={|ε−ε0|/ε0}, which is a ratio of an absolute value |ε−ε0| of a deviation amount of the measured (i.e., actually measured) strain amount ε from the assumed strain amount ε0 with respect to the assumed strain amount ε0, is within a preset strain allowable value (for example, 0.05, that is, 5% by percentage) (S8). The strain allowable value, for example, may not be 5%. In a case where the above-described strain ratio R is within the strain allowable value, the control unit 52 determines that the compliance value of the patient's aneurysm is approximate to the compliance value (reference compliance value) of a general aneurysm, and determines to suggest a coil having the specifications (for example, the loop diameter and the coil length) of the embolic coil selected from the image (S9). Next, the control unit 52 causes the display unit 53 to display indication that the coil selected from the image is used as the coil is currently configured (S14). Usually, in a procedure of embolizing an aneurysm, a framing coil is packed around an aneurysm wall, then a filling coil is packed, and finally a finishing coil is packed. Each embolic coil can be cut, for example, by passing a current through the embolic coil. The configuration of the embolic coil is not limited to the configurations disclosed above.

In a case where the above-described strain ratio R exceeds the strain allowable value, the control unit 52 determines that the actual compliance value of the aneurysm of the patient being treated deviates from the reference compliance value of the general aneurysm, and calculates the actual compliance value (S10). The compliance value is calculated as a strain difference value (ε−ε0) obtained by subtracting the assumed strain amount ε0 from the strain amount ε, or a value to which the difference value is applied.

The control unit 52 determines whether the calculated compliance value is greater than a reference compliance value of a preset general aneurysm (S11). In a case where the calculated compliance value is greater than the reference compliance value, the control unit 52 determines that the patient's aneurysm is relatively flexible and rather easy to stretch, and determines to suggest a coil having a larger loop diameter and/or longer coil length than the loop diameter and/or coil length of the embolic coil selected from the image (S12). That is, in a case where the aneurysm is relatively flexible and rather easy to stretch, the control unit 52 performs a suggestion such that a high coil packing ratio can be obtained even when some force is applied to the aneurysm. The coil packing ratio is a ratio of the volume occupied by the embolic coil inside the aneurysm. Next, the control unit 52 causes the display unit 53 to display the coil determined to be used (S14). Alternatively, in S12, the control unit 52 may suggest changing the treatment method instead of changing the suggested embolic coil from the embolic coil selected from the image. For example, the control unit 52 may determine to suggest packing the inside of the aneurysm with the coil in the coil packing ratio (for example, 40% or more) higher than the normal coil packing ratio (for example, 30%), and display the result on the display unit 53 in S14.

In a case where the calculated compliance value is equal to or less than the reference compliance value, the control unit 52 determines that the patient's aneurysm being subjected to the treatment is relatively hard and rather difficult to stretch, and determines to suggest a coil having a smaller loop diameter and/or shorter coil length than the loop diameter and/or coil length of the embolic coil selected from the image (S13). That is, in a case where the aneurysm is relatively hard and rather difficult to stretch, the control unit 52 performs a suggestion that a low coil packing ratio can be obtained such that the coil is not excessively packed in the aneurysm. Next, the control unit 52 causes the display unit 53 to display that the coil determined to be used is used to treat the aneurysm (S14). Alternatively, in S13, the control unit 52 may suggest changing the treatment method instead of changing the suggested embolic coil from the embolic coil selected from the image. For example, the control unit 52 may determine and suggest packing the inside of the aneurysm with the coil in the coil packing ratio (for example, 15% to 20%) lower than the normal coil packing ratio (for example, 30%), and display the result on the display unit 53 in S14.

Next, the control unit 52 controls the inflation control device 70 to discharge the fluid from the balloon 12, and deflates the balloon 12 (S15). Subsequently, the operator removes the detection device 10 from the blood vessel and ends the examination.

Next, the operator prepares the suggested coil and packs the inside of the aneurysm with the coil. Alternatively, in a case where the packing ratio is suggested, the operator packs a framing coil, a filling coil, and a finishing coil so as to obtain the packing ratio, and completes the treatment.

As described above, according to the present embodiment, the medical support system 1 that supports intravascular treatment includes: the detection device 10 inserted into a blood vessel and configured to be capable of detecting flexibility of a treatment target which is the blood vessel or an object (for example, a thrombus) attached to the blood vessel; and the control unit 52 configured to select a treatment device (for example, an embolic coil) or a treatment method, which is suitable for the flexibility of the treatment target detected by the detection device 10.

In the medical support system 1 configured as described above, the state of the blood vessel or the state of the object attached to the blood vessel is quantified, and the control unit 52 selects an appropriate treatment device or treatment method according to the numerical value. Therefore, the medical support system 1 can select the appropriate treatment device or treatment method according to the blood vessel in a different state or the object attached to the blood vessel.

The detection device 10 includes the inflation portion (for example, the balloon 12) capable of inflating and deflating in the blood vessel, the contact detection unit 20 configured to detect contact of the inflation portion with the treatment target, and the strain detection unit 30 configured to detect a strain amount E of the treatment target. The control unit 52 receives a contact detection result from the contact detection unit 20 and a strain amount detection result from the strain detection unit 30, calculates flexibility or elasticity which is a state of the treatment target from the contact detection result and the strain amount detection result, and selects the treatment device or treatment method on the basis of the calculated flexibility or elasticity. Therefore, the medical support system 1 can relatively accurately detect the flexibility of the treatment target, and thus it is possible to select an appropriate treatment device or treatment method.

The control unit 52 is capable of receiving (i.e., configured to receive) an image from the imaging apparatus 60, which is configured to capture the image with which the size of the treatment target can be specified. The control unit 52 determines whether or not a strain ratio R={|ε−ε0|/ε0}, which is a ratio of an absolute value |ε−ε0| of a deviation amount of an actual strain amount ε detected by the strain detection unit 30, with respect to an assumed strain amount ε0 which is a strain amount assumed to be generated in the treatment target in a case where a predetermined inflation force is applied to the inflation portion, is within a preset allowable value on the assumption that the treatment target includes a reference compliance value set in advance, and determines the treatment device or treatment method calculated from the size of the treatment target specified from the image acquired from the imaging apparatus 60 as a treatment device or treatment method to be selected, in a case where the strain ratio R is within the allowable value; and the control unit 52 calculates an actual compliance value of the treatment target from the strain amount ε measured by the strain detection unit 30 to determine whether the compliance value is greater than or less than the reference compliance value in a case where a predetermined inflation force is applied to the inflation portion after the contact detection unit 20 detects the contact of the inflation portion with the treatment target in a case where the strain ratio R exceeds the allowable value, determines a treatment device or treatment method capable of obtaining a coil packing ratio higher than the coil packing ratio of the treatment device or treatment method calculated from the size of the treatment target specified from the image acquired from the imaging apparatus 60, as a treatment device or treatment method to be selected, in a case where the actual compliance value is greater than the reference compliance value, and determines a treatment device or treatment method capable of obtaining a coil packing ratio lower than the coil packing ratio of the treatment device or treatment method calculated from the size of the treatment target specified from the image acquired from the imaging apparatus 60, as a treatment device or treatment method to be selected, in a case where the actual compliance value is less than the reference compliance value. Therefore, the medical support system 1 can select an appropriate treatment device or treatment method by using both the size of the treatment target that can be specified from the image obtained from the imaging apparatus 60 and the measured compliance value of the treatment target. The present medical support system 1 can determine a treatment method such as determining that the high coil packing ratio can be obtained even when some force is applied in a case where the actual compliance value is relatively great and the treatment target is relatively soft, or performing determination that the low coil packing ratio can be obtained such that the coil is not excessively packed in a case where the actual compliance value is relatively small and the treatment target is relatively hard.

The method for controlling a medical support system 1 that supports intravascular treatment can include: receiving a detection result from the detection device 10 inserted into a blood vessel and configured to be capable of detecting a state of a treatment target which is the blood vessel or an object attached to the blood vessel; selecting a treatment device or treatment method suitable for the state of the treatment target detected by the detection device 10; and transmitting information regarding the selected treatment device or treatment method to the display unit 53 capable of displaying the information to cause the display unit 53 to display the information.

In the method for controlling a medical support system 1 configured as described above, the state of the blood vessel or the state of the object attached to the blood vessel can be quantified, and an appropriate treatment device or treatment method according to the numerical value can be selected and displayed on the display unit 53. Therefore, in the method for controlling a medical support system 1, the appropriate treatment device or treatment method according to the blood vessel in a different state or the object attached to the blood vessel can be selected and displayed on a display unit.

The receiving of the detection result can include receiving a contact detection result from the contact detection unit 20 configured to detect contact of the inflation portion capable of inflating and deflating in the blood vessel with the treatment target, and receiving a strain amount detection result from the strain detection unit 30 configured to detect the strain amount ε of the treatment target, and the selecting of the treatment device or treatment method includes calculating flexibility or elasticity which is a state of the treatment target from the contact detection result and the strain amount detection result, and selecting the treatment device or treatment method on the basis of the calculated flexibility or elasticity. Therefore, the method for controlling a medical support system 1 can relatively accurately detect the flexibility or elasticity of the treatment target, and thus it is possible to control the medical support system 1 so as to select an appropriate treatment device or treatment method.

The selecting of the treatment device or treatment method can include: determining whether or not a strain ratio R={|ε−ε0|/ε0}, which is a ratio of an absolute value |ε−ε0| of a deviation amount of an actual strain amount ε detected by the strain detection unit 30, with respect to an assumed strain amount ε0 which is a strain amount assumed to be generated in the treatment target in a case where a predetermined inflation force is applied to the inflation portion, is within a preset allowable value on an assumption that the treatment target includes a reference compliance value set in advance; determining a treatment device or treatment method calculated from a size of the treatment target specified from an image acquired from the imaging apparatus 60 capable of capturing the image of the treatment target as a treatment device or treatment method to be selected, in a case where the strain ratio R is within the allowable value; calculating an actual compliance value of the treatment target from the strain amount ε measured by the strain detection unit 30 to determine whether the compliance value is greater than or less than the reference compliance value when a predetermined inflation force is applied to the inflation portion in a case where the strain ratio R exceeds the allowable value; determining a treatment device or treatment method capable of obtaining a coil packing ratio higher than the coil packing ratio of the treatment device or treatment method calculated from the size of the treatment target specified from the image acquired from the imaging apparatus 60, as a treatment device or treatment method to be selected, in a case where the actual compliance value is greater than the reference compliance value; and determining a treatment device or treatment method capable of obtaining a coil packing ratio lower than the coil packing ratio of the treatment device or treatment method calculated from the size of the treatment target specified from the image acquired from the imaging apparatus 60, as a treatment device or treatment method to be selected, in a case where the actual compliance value is less than the reference compliance value. Therefore, the method for controlling a medical support system 1 can control the medical support system 1 so as to select an appropriate treatment device or treatment method by using both the size of the treatment target that can be specified from the image obtained from the imaging apparatus 60 and the measured compliance value of the treatment target. In the present control method, a treatment method can be determined, such as performing determination that the high coil packing ratio can be obtained even when some force is applied in a case where the actual compliance value is relatively great and the treatment target is relatively soft, or performing determination that the low coil packing ratio can be obtained such that the coil is not excessively packed in a case where the actual compliance value is relatively small and the treatment target is relatively hard.

The medical support method that supports intravascular treatment includes: inserting, into a blood vessel, the detection device 10 capable of detecting a state of a treatment target which is the blood vessel or an object attached to the blood vessel to detect the state of the treatment target; selecting a treatment device or treatment method suitable for the state of the treatment target detected by the detection device 10; and displaying information regarding the selected treatment device or treatment method on the display unit 53 capable of displaying the information.

In the medical support method configured as described above, the state of the blood vessel or the state of the object attached to the blood vessel is quantified, and an appropriate treatment device or treatment method according to the numerical value can be selected to be displayed on the display unit 53. Therefore, in the medical support method, the appropriate treatment device or treatment method according to the blood vessel in a different state or the object attached to the blood vessel can be selected to be displayed.

The detecting of the state of the treatment target includes detecting contact of the inflation portion capable of inflating and deflating in the blood vessel with the treatment target and detecting the strain amount ε of the treatment target, and the selecting of the treatment device or treatment method includes calculating flexibility or elasticity which is a state of the treatment target from the contact detection result and the strain amount detection result, and selecting the treatment device or treatment method on the basis of the calculated flexibility or elasticity. Therefore, in the medical support method, the flexibility or elasticity of the treatment target can be accurately detected, and thus it is possible to select an appropriate treatment device or treatment method.

The selecting of the treatment device or treatment method includes: determining whether or not a strain ratio R={|ε−ε0|/ε0}, which is a ratio of an absolute value |ε−ε0| of a deviation amount of an actual strain amount ε detected by the strain detection unit 30, with respect to an assumed strain amount ε0 which is a strain amount assumed to be generated in the treatment target in a case where a predetermined inflation force is applied to the inflation portion, is within a preset allowable value on an assumption that the treatment target includes a reference compliance value set in advance; determining a treatment device or treatment method calculated from a size of the treatment target specified from an image acquired from the imaging apparatus 60 capable of capturing the image of the treatment target as a treatment device or treatment method to be selected, in a case where the strain ratio R is within the allowable value; calculating an actual compliance value of the treatment target from the strain amount ε measured by the strain detection unit 30 to determine whether the compliance value is greater than or less than the reference compliance value when a predetermined inflation force is applied to the inflation portion in a case where the strain ratio R exceeds the allowable value; determining a treatment device or treatment method capable of obtaining a coil packing ratio higher than the coil packing ratio of the treatment device or treatment method calculated from the size of the treatment target specified from the image acquired from the imaging apparatus 60, as a treatment device or treatment method to be selected, in a case where the actual compliance value is greater than the reference compliance value; and determining a treatment device or treatment method capable of obtaining a coil packing ratio lower than the coil packing ratio of the treatment device or treatment method calculated from the size of the treatment target specified from the image acquired from the imaging apparatus 60, as a treatment device or treatment method to be selected, in a case where the actual compliance value is less than the reference compliance value. Therefore, in the medical support method, an appropriate treatment device or treatment method can be selected by using both the size of the treatment target that can be specified from the image obtained from the imaging apparatus 60 and the measured compliance value of the treatment target. In the present medical support method, a treatment method can be determined, such as performing determination that the high coil packing ratio can be obtained even when some force is applied in a case where the actual compliance value is great and the treatment target is soft, or performing determination that the low coil packing ratio can be obtained such that the coil is not excessively packed in a case where the actual compliance value is relatively small and the treatment target is relatively hard.

The present disclosure is not limited to the above-described embodiment, and various modifications can be made by those skilled in the art within the technical idea of the present disclosure. For example, the medical support system 1 according to the above-described embodiment may be applied to other than the cerebral aneurysm. That is, the treatment target may be an aneurysm other than the cerebral aneurysm, a blood vessel wall other than that of the aneurysm, a thrombus attached to the blood vessel, or the like. The medical support system 1 according to the embodiment can quantify the vulnerability of the blood vessel in the aneurysm or blood vessel wall or the viscosity of the thrombus attached to the blood vessel, and select a treatment device or a treatment method having an optimum size or shape according to the numerical value.

The treatment device selected by the medical support system 1 is not limited to the embolic coil, and may be, for example, a flow diverter stent, a stent retriever, or the like. The medical support system 1 may be used for selection of the treatment method. For example, the medical support system 1 may detect a state of the thrombus (an object attached to the blood vessel) which is the treatment target and select an optimal aspiration force for aspirating and removing the thrombus. Therefore, by using the selected treatment method, it is possible to obtain an effect of reducing the insufficient removal of the thrombus and an effect of reducing the risk of giving a burden to the blood vessel wall due to excessive aspiration.

The inflation portion may not be the balloon 12. The strain amount ε may not be obtained from the strain sensor 31 disposed in the balloon 12 but may be calculated from a change in size of the treatment target specified from the image obtained from the imaging apparatus 60.

The detailed description above describes embodiments of a medical support system that supports selection of a device and a treatment method, which are appropriate for intravascular treatment, a method of controlling a medical support system, and a medical support method. The invention is not limited, however, to the precise embodiments and variations described. Various changes, modifications and equivalents may occur to one skilled in the art without departing from the spirit and scope of the invention as defined in the accompanying claims. It is expressly intended that all such changes, modifications and equivalents which fall within the scope of the claims are embraced by the claims.

What is claimed is:

1. A medical support system that supports intravascular treatment, the medical support system comprising:
a detection device inserted into a blood vessel and configured to detect a state of a treatment target, the treatment target being the blood vessel or an object attached to the blood vessel; and
a control unit configured to select a treatment device or a treatment method, is the treatment device or the treatment method being suitable for the state of the treatment target detected by the detection device.

2. The medical support system according to claim 1, wherein the detection device comprises:
an inflation portion configured to inflate and deflate in the blood vessel;
a contact detection unit configured to detect contact of the inflation portion with the treatment target; and
a strain detection unit configured to detect a strain amount of the treatment target.

3. The medical support system according to claim 2, wherein the control unit is further configured to:
receive a contact detection result from the contact detection unit and a strain amount detection result from the strain detection unit;
calculate flexibility or elasticity, which is a state of the treatment target, from the contact detection result and the strain amount detection result; and
select the treatment device or the treatment method on a basis of the calculated flexibility or elasticity.

4. The medical support system according to claim 3, wherein the control unit is further configured to:
receive an image from an imaging apparatus configured to capture the image with which a size of the treatment target can be specified;
determine whether or not a strain ratio, which is a ratio of an absolute value of a deviation amount of an actual strain amount detected by the strain detection unit, with respect to an assumed strain amount which is a strain amount assumed to be generated in the treatment target in a case where a predetermined inflation force is applied to the inflation portion, is within a preset allowable value on an assumption that the treatment target includes a reference compliance value set in advance; and
determine the treatment device or the treatment method calculated from the size of the treatment target specified from the image acquired from the imaging apparatus as a treatment device or the treatment method to be selected, in a case where the strain ratio is within the allowable value.

5. The medical support system according to claim 4, wherein the control unit is further configured to:
calculate an actual compliance value of the treatment target from the strain amount measured by the strain detection unit to determine whether the compliance value is greater than or less than the reference compliance value in a case where a predetermined inflation force is applied to the inflation portion after the contact detection unit detects the contact of the inflation portion with the treatment target in a case where the strain ratio exceeds the allowable value; and
determine a treatment device or a treatment method configured to obtain a coil packing ratio higher than the coil packing ratio of the treatment device or the treatment method calculated from the size of the treatment target specified from the image acquired from the imaging apparatus, as the treatment device or the treatment method to be selected, in a case where the actual compliance value is greater than the reference compliance value.

6. The medical support system according to claim 4, wherein the control unit is configured to:
calculate an actual compliance value of the treatment target from the strain amount measured by the strain detection unit to determine whether the compliance value is greater than or less than the reference compliance value in a case where a predetermined inflation force is applied to the inflation portion after the contact detection unit detects the contact of the inflation portion with the treatment target in a case where the strain ratio exceeds the allowable value; and
determine a treatment device or a treatment method configured to obtain a coil packing ratio lower than the coil packing ratio of the treatment device or the treatment method calculated from the size of the treatment target specified from the image acquired from the imaging apparatus, as the treatment device or the treatment method to be selected, in a case where the actual compliance value is less than the reference compliance value.

7. The medical support system according to claim 1, further comprising:
a display unit configured to display the selected treatment device or the selected treatment method by the control unit.

8. A method for controlling a medical support system that supports intravascular treatment, the method comprising:
receiving a detection result from a detection device inserted into a blood vessel and configured to be capable of detecting a state of a treatment target which is the blood vessel or an object attached to the blood vessel;
selecting a treatment device or a treatment method suitable for the state of the treatment target detected by the detection device; and
transmitting information regarding the selected treatment device or the selected treatment method to a display unit configured to display the information.

9. The method for controlling the medical support system according to claim 8, further comprising:
displaying the selected treatment device or the selected treatment method on the display unit.

10. The method for controlling the medical support system according to claim 8, wherein the receiving of the detection result further comprises:
receiving a contact detection result from a contact detection unit configured to detect contact of an inflation portion capable of inflating and deflating in the blood vessel with the treatment target; and
receiving a strain amount detection result from a strain detection unit configured to detect a strain amount of the treatment target.

11. The method for controlling the medical support system according to claim 10, wherein the selecting of the treatment device or the treatment method comprises:
calculating flexibility or elasticity which is a state of the treatment target from the contact detection result and the strain amount detection result; and
selecting the treatment device or the treatment method on a basis of the calculated flexibility or elasticity.

12. The method for controlling a medical support system according to claim 11, wherein the selecting of the treatment device or the treatment method comprises:
determining whether or not a strain ratio, which is a ratio of an absolute value of a deviation amount of an actual strain amount detected by the strain detection unit, with respect to an assumed strain amount which is a strain amount assumed to be generated in the treatment target in a case where a predetermined inflation force is applied to the inflation portion, is within a preset allowable value on an assumption that the treatment target includes a reference compliance value set in advance; and
determining a treatment device or a treatment method calculated from a size of the treatment target specified from an image acquired from an imaging apparatus configured to capture the image of the treatment target as a treatment device or treatment method to be selected, in a case where the strain ratio is within the allowable value.

13. The method for controlling a medical support system according to claim 12, further comprising:
calculating an actual compliance value of the treatment target from the strain amount measured by the strain detection unit to determine whether the compliance value is greater than or less than the reference compliance value when a predetermined inflation force is applied to the inflation portion in a case where the strain ratio exceeds the allowable value; and
determining a treatment device or a treatment method configured to obtain a coil packing ratio higher than the coil packing ratio of the treatment device or the treatment method calculated from the size of the treatment target specified from the image acquired from the imaging apparatus, as a treatment device or a treatment method to be selected, in a case where the actual compliance value is greater than the reference compliance value.

14. The method for controlling a medical support system according to claim 12, further comprising:
calculating an actual compliance value of the treatment target from the strain amount measured by the strain detection unit to determine whether the compliance value is greater than or less than the reference compliance value when a predetermined inflation force is applied to the inflation portion in a case where the strain ratio exceeds the allowable value; and
determining a treatment device or a treatment method configured to obtain a coil packing ratio lower than the coil packing ratio of the treatment device or the treatment method calculated from the size of the treatment target specified from the image acquired from the imaging apparatus, as a treatment device or a treatment method to be selected, in a case where the actual compliance value is less than the reference compliance value.

15. A medical support method that supports intravascular treatment, the method comprising:
inserting, into a blood vessel, a detection device configured to be capable of detecting a state of a treatment target which is the blood vessel or an object attached to the blood vessel to detect flexibility of the treatment target;
selecting a treatment device or treatment method suitable for the state of the treatment target detected by the detection device; and
displaying information regarding the selected treatment device or the treatment method on a display unit.

16. The medical support method according to claim 15, wherein the detecting of the state of the treatment target further comprises:
detecting contact of an inflation portion capable of inflating and deflating in the blood vessel with the treatment target; and
detecting a strain amount of the treatment target.

17. The medical support method according to claim 16, wherein the selecting of the treatment device or treatment method further comprises:
calculating flexibility or elasticity which is the state of the treatment target from a contact detection result and a strain amount detection result; and
selecting the treatment device or treatment method on a basis of the calculated flexibility or elasticity.

18. The medical support method according to claim 17, wherein the selecting of the treatment device or treatment method comprises:
determining whether or not a strain ratio, which is a ratio of an absolute value of a deviation amount of an actual strain amount detected by a strain detection unit, with respect to an assumed strain amount which is a strain amount assumed to be generated in the treatment target in a case where a predetermined inflation force is applied to the inflation portion, is within a preset allowable value on an assumption that the treatment target includes a reference compliance value set in advance; and
determining a treatment device or treatment method calculated from a size of the treatment target specified from an image acquired from an imaging apparatus configured to be capable of capturing the image of the treatment target as a treatment device or treatment method to be selected, in a case where the strain ratio is within the allowable value.

19. The medical support method according to claim 18, further comprising:
calculating an actual compliance value of the treatment target from the strain amount measured by the strain detection unit to determine whether the compliance value is greater than or less than the reference compliance value when a predetermined inflation force is applied to the inflation portion in a case where the strain ratio exceeds the allowable value and
determining a treatment device or treatment method configured to obtain a coil packing ratio higher than the coil packing ratio of the treatment device or treatment method calculated from the size of the treatment target specified from the image acquired from the imaging apparatus, as a treatment device or treatment method to be selected, in a case where the actual compliance value is greater than the reference compliance value.

20. The medical support method according to claim 18, further comprising:
calculating an actual compliance value of the treatment target from the strain amount measured by the strain detection unit to determine whether the compliance value is greater than or less than the reference compliance value when a predetermined inflation force is applied to the inflation portion in a case where the strain ratio exceeds the allowable value; and
determining a treatment device or treatment method configured to obtain a coil packing ratio lower than the coil packing ratio of the treatment device or treatment method calculated from the size of the treatment target specified from the image acquired from the imaging apparatus, as a treatment device or treatment method to be selected, in a case where the actual compliance value is less than the reference compliance value.
